(19) United States
(12) Patent Application Publication
(10) Pub. No.: US 2006/0188877 A1
(43) Pub. Date: Aug. 24, 2006

(54) Fluorescence-quenching used to detect nucleic acid oligomer hybrization events at high salt concentrations

(76) Inventors: Gerhard Hartwich, Munchen (DE); Thomas Kratzmuller, Munchen (DE); Herbert Wieder, Mannheim (DE)

Correspondence Address: CROCKETT & CROCKETT, 24012 CALLE DE LA PLATA, SUITE 400, LAGUNA HILLS, CA, 92653, US

(21) Appl. No.: 10/529,472

(22) PCT Filed: Nov. 8, 2002
(86) PCT No.: PCT/DE02/04148

§ 371 Date: Feb. 21, 2006

(30) Foreign Application Priority Data
Nov. 9, 2001 (DE) 10155055.3

(51) Int. Cl. C12Q 1/68

(52) U.S. Cl. 435/6

(57) ABSTRACT

A method is described for detecting nucleic acid oligomer hybridization events by fluorescence quenching, which comprises as a first step the provision of a modified surface. The modification of the surface consists in the binding of at least one type of modified nucleic acid oligomers 201, wherein said nucleic acid oligomers 201 are modified by at least one type of fluorophore 102 bound to it. The further steps of the inventive method are: providing a sample that includes nucleic acid oligomers, contacting said sample with the modified surface, adjusting a defined salt concentration of greater than 0.5 mol/l in the solution surrounding the modified nucleic acid oligomers, detecting the fluorescence of the fluorophore and comparing the detected fluorescence intensity with reference values.

### FIELD OF THE INVENTION

The present invention is directed to a method for detecting nucleic acid oligomer hybridization events by fluorescence quenching.

### STATE OF THE ART

Immunoassays and increasingly also DNA and RNA sequence analysis are used for disease diagnosis, toxicological test procedures, genetic research and development, as well as in agricultural and pharmaceutical sectors. Besides the known serial methods with autoradiographical or optical detection parallel detection methods by means of array technology, so called DNA or protein chips, are increasingly used. The detection of these parallel techniques is also based on optical, autoradiographical or electrochemical methods.

For gene analysis on a chip a set of known DNA sequences (“probe oligonucleotides”) are located in an ordered grid with the position of each individual DNA sequence being known. Fragments of active genes present in the analyzing solution whose sequences are complementary to certain probe oligonucleotides on the chip can be identified by the detection of the corresponding hybridization event. Generally the analysis is carried out with optical (or autoradiographical) detection techniques using target oligonucleotides labeled with a radiolabel (e.g. ³²P) or a fluorescent dye (e.g. fluorescein, Cy3™ or Cy5™). The use of fluorescent labels and corresponding fluorescence scanners increasingly entails the use of radioactive labels. Fluorescence scanners currently available on the market allow the detection of fluorescent dyes in the sub-attomol range.

The use of labeled targets for the detection of hybridization events is associated with several disadvantages. First the labeling has to be carried out before the measurement in fact, which means an additional synthesis step and therewith additional expenditure of human labor. Furthermore it is difficult to ensure a homogeneous labeling of the simple material. Moreover stringent washing conditions are necessary to remove unbound or non-specifically bound material subsequent to the hybridization.

For the analysis of both proteins and DNA it is therefore desirable and beneficial for the user, if targets (antibodies, antigens or DNA fragments respectively) need not be modified with a detection label.

The disadvantages of labeling the sample material with radioactive elements or fluorescent dyes can be avoided, if instead of the targets the probe molecules are labeled with appropriate fluorescent dyes. So called molecular beacons work according to this principle. These single stranded oligonucleotides possess a hairpin (stem-and-loop) structure and are tagged with a Fluorophor (e.g. Fluoresceine, TexasRed®) at one end of the sequence and a suitable fluorescence quencher (e.g. DABCYL) at the other end. Through the special geometric arrangement the fluorescent group and the unit leading to the quenching of the fluorescence are located in close sterical proximity. Therefore the probes show very little fluorescence. In the presence of appropriate sequences (target) complimentary to the sequence of the loop hybridization occurs in that region. This leads to a conformational change and to a separation of fluorophor and quencher, which can be observed as a strong increase in fluorescence.

Besides organic molecules (such as DABCYL) also gold nanoparticles are used as efficient quencher (cf. Nature Biotech. Vol. 19, 2001, page 365). The quenching of the fluorescence by metals is primarily based on a radiationless energy transfer from the dye to the metal. When using gold nanoparticles a greater sensitivity can be seen compared to organic quencher. Furthermore dyes are quenched up to the near infrared range. But a disadvantage of this technology is due to the fact that gold nanoparticles are no longer stable at temperature above 50° C. Another disadvantage underlies the fact that this method is limited to the analysis of solutions and therefore only a few sequences can be analyzed at the same time, thus the degree of parallelization is low.

Known in the art are also examinations for the detection of nucleic acid oligomer hybridization events by means of fluorescence quenching (T. Neumann, Dissertation “Strategies for Detecting DNA Hybridisation Using Surface Plasmon Fluorescence Spectroscopy”, Mainz, June 2001). This research work shows that the concentration of salt in the surrounding solution has an influence on the conformation of the nucleic acid oligomers. An increase of fluorescence intensity by a factor of 1.75 was observed after hybridization, which leads to an unsatisfying detection limit particularly when using parallel methods.

Even though there are many possibilities for the detection of nucleic acid oligomer hybridization events, there is a high demand for simple, cost-efficient, trouble-free and reliable detection principles primarily in the field of lower density array technologies (DNA and protein chips with few singular up to several hundreds of thousands test sites per cm² e.g. for so called POC (Point of care) systems and for high throughput screening (HTS) systems respectively.

### DESCRIPTION OF THE INVENTION

The task of the present invention is to provide a method for the detection of nucleic acid oligomer hybrids that does not exhibit the disadvantages of the background art.

According to the present invention this task is fulfilled by the method as stated in independent claim 1 and by the kit as stated in independent claim 11.

Further advantageous details, aspect and embodiments of the present invention follow from the dependent claims, the description, the figures and the examples.

The following abbreviations and terms will be used in the context of the present invention:

Genetics

| | |
|---|---|
| DNA | Deoxyribonucleic acid |
| RNA | Ribonucleic acid |
| PNA | Peptide nucleic acid (synthetic DNA or RNA in which the sugar- |
| | phosphate moiety is replaced by an amino acid. If the sugar- |
| | phosphate moiety is replaced by the —NH—(CH₂)₂—N(COCH₂— |
| | base)-CH₂CO-moiety, PNA will hybridize with DNA.) |
| A | Adenine |
| G | Guanine |
| C | Cytosine |
| T | Thymine |
| U | Uracil |
| base | A, G, T, C or U |
| bp | Base pair |
| nucleic acid | At least two covalently joined nucleotides or at least two |
| | covalently joined pyrimidine (e.g. cytosine, thymine, or uracil) or |
| | purine bases (e.g. adenine or guanine). The term nucleic acid |
| | refers to any backbone of the covalently linked pyrimidine or |
| | purine bases, such as the sugar-phosphate backbone of DNA, |
| | cDNA, or RNA, a peptide backbone of PNA, or analogous |
| | structures (e.g. a phosphoramide, thiophosphate, or |
| | dithiophosphate backbone). The essential feature of a nucleic |
| | acid according to the present invention is that it can sequence- |
| | specifically bind naturally occurring cDNA or RNA. |
| nt | Nucleotide |
| nucleic acid oligomer | Nucleic acid of a base length that is not further specified (e.g. |
| | nucleic acid octamer: a nucleic acid having any backbone in |
| | which 8 pyrimidine or purine bases are covalently bound to one |
| | another). |
| ns oligomer | Nucleic acid oligomer |
| oligomer | Equivalent to nucleic acid oligomer. |
| oligonucleotide | Equivalent to oligomer or nucleic acid oligomer, e.g. a DNA, |
| | PNA, or RNA fragment of a base length that is not further |
| | specified. |
| oligo | Abbreviation for oligonucleotide. |
| mismatch | To form the Watson-Crick double-stranded oligonucleotide |
| | structure, the two single-strands hybridize in such a way that the |
| | A (or C) base of one strand forms hydrogen bonds with the T (or |
| | G) base of the other strand (in RNA, T is replaced by uracil). Any |
| | other base pairing does not form hydrogen bonds, distorts the |
| | structure and is referred to as a “mismatch.” |
| ss | Single-strand |
| ds | double-strand |

Chemical substances/Groups

| | |
|---|---|
| fluorophore | Chemical compound (chemical substance) that has the ability to |
| | emit fluorescence light of longer wavelength (red shifted) on |
| | excitation with light. Fluorophores (fluorescent dyes) can absorb |
| | light in a wavelength range from the ultraviolet (UV) through the |
| | visible up to the infrared range. The emission maxima is |
| | normally shifted by 15 to 40 nm compared to the maxima of |
| | absorption (Stokes shift) |
| FP | Fluorophore |
| Cy3 ™ | 5,5′-disulfo-1,1′di(-carbopentenyl)-3,3,3′,3′-tetramethyl- |
| | indodicarbocyanine |
| | (fluorescent dye of Amersham Life Science, Inc.) |
| Cy5 ™ | 5,5′,7,7′-tetrasulfo-1,1′di(-carbopentenyl)-3,3,3′,3′-tetramethyl- |
| | benzindodicarbocyanine |
| | (fluorescent dye of Amersham Life Science, Inc.) |
| fluoresceine | resorcinolphtalein (fluorescent dye) |
| Rhodamin 6G | Basic Red 1 (fluorescent dye) |
| Texas Red ® | fluorescent dye of Molecular Probes, Inc. |
| DABCYL | 4-((4′-(Dimethylamino)phenyl)azo) benzoic acid |
| Fluorescence | Radiationless energy transfer |
| quenching | |
| quenching surface | Conducting (metal) surface that has the ability to quench |
| | fluorescence by energy transfer (in particular gold, silver, copper |
| | surfaces etc.) |
| EDTA | ethylenediamine tetraacetate (sodium salt) |
| linker | A molecular link between two molecules or between a surface |
| | atom, surface molecule, or surface molecule group and another |
| | molecule. Linkers can usually be purchased in the form of alkyl, |
| | alkenyl, alkynyl, heteroalkyl, heteroalkenyl, or heteroalkynyl |
| | chains, the chain being derivatized in two places with (identical |
| | or different) reactive groups. These groups form a covalent |
| | chemical bond in simple/known chemical reactions with the |
| | appropriate reaction partners. The reactive groups may also be |
| | photoactivatable, i.e. the reactive groups are activated only by |
| | light of a specific or any given wavelength. Preferred linkers are |
| | those having a chain length of 1-20, especially a chain length |
| | of 1-14, the chain length here representing the shortest |
| | continuous link between the structures to be joined, in other |
| | words between the two molecules or between a surface atom, |
| | surface molecule, or surface molecule group and another |
| | molecule. |
| spacer | A linker that is covalently attached via the reactive groups to one |
| | or both of the structures to be joined (see linker). Preferred |
| | spacers are those having a chain length of 1-20, especially a chain |
| | length of 1-14, the chain length representing the shortest |
| | continuous link between the structures to be joined. |

Modified Surfaces/Electrodes

| | |
|---|---|
| Au—S—(CH₂)₂- | Gold film having a covalently applied monolayer of derivatized |
| ss-oligo-FP | single-strand DNA oligonucleotide. Here, the oligonucleotide's |
| | terminal phosphate group at the 3′-end is esterified with (HO—(CH₂)₂— |
| | S)₂ to form P—O—(CH₂)₂—S—S—(CH₂)₂—OH, the S—S bond being |
| | homolytically cleaved and producing one Au—S—R bond each. At the |
| | free 5′-end of the probe oligonucleotide a fluorophor (FP) such as |
| | Cy3 ™, Cy5 ™, Texas Red ®, Rhodamin 6G, fluoresceine etc. is |
| | covalently attached. |
| Au—S—(CH₂)₂- | Au—S—(CH₂)₂-ss-oligo-FP hybridized with the oligonucleotide that is |
| ds-oligo-FP | complementary to the ss-oligo |

The present invention is directed to a method for detecting nucleic acid oligomer hybridization events by fluorescence quenching that includes as a first step the preparation of a modified surface. The modification of the surface comprises of the binding of at least one type of modified nucleic acid oligomer, where the nucleic acid oligomer is modified by the attachment of at least one type of fluorophor. The further steps of the method according to the present invention are the provision of a sample with nucleic acid oligomers, the exposure of the sample to the modified surface, the adjusting of a defined salt concentration higher than 0.5 mol/l in the solution surrounding the modified nucleic acid oligomers, the detection of the fluorescence of the fluorophore and the comparison of the fluorescence intensity obtained during detection with reference values

Within the method according to the invention it is necessary to compare the detected fluorescence intensities with reference values. These reference values can exist from earlier measurements and therefore in the most general case they have not to be detected during the method according to the invention. But, since in the ideal case the reference values should be obtained on exactly the same external conditions as the actual values of the fluorescence intensity, according to a preferred embodiment of the present invention, a first detection of the fluorescence of the fluorophor is carried out before the exposure of the target (sample) to the modified surface. For that purpose a defined salt concentration is adjusted in the solution surrounding the modified nucleic acid oligomers with the same salt concentration being used as for the second detection of the fluorescence of the Fluorophor, followed by the first detection of the fluorescence of the Fluorophor.

Subsequently stringent conditions are set for the hybridization and the sample will be brought in contact with the modified surface. The values obtained with this first fluorescence detection are used as reference values and compared with the values obtained with the second detection of the fluorescence.

The setting of the stringent conditions for the hybridization and the exposure of the sample to the modified surface can in principle be accomplished in any chronological order. Preferably the setting of the stringent conditions for the hybridization is done simultaneously with the exposure of the sample to the modified surface or is carried out after bringing the sample in contact with the modified surface.

According to the subsequently described particularly preferable embodiments of the present invention an additional measurement is carried out for scaling. In such cases sites are applied to the modified surface, to which a clearly defined degree of association can be assigned after addition of the sample. The signal obtained with the detection is characteristical for this certain degree of association and can be used for scaling the test sites.

The present invention namely includes also methods, where a modified surface is used, which has been modified by binding at least two types of modified nucleic acid oligomers. The different types of modified nucleic acid oligomers are bound to the surface in spatial basically separated areas. The term “spatial basically separated areas” is understood to mean areas on the surface that are predominantly modified by binding of a certain type of modified nucleic acid oligomer. Solely in areas, where two of such basically separated areas adjoin, a spatial mixture of different types of modified nucleic acid oligomers may occur.

In the preferred method in the context of the present invention a nucleic acid oligomer is added to the sample before the exposure of the sample to the modified surface, with the nucleic acid oligomer being a binding partner with a high association constant for a certain type of modified nucleic acid oligomer, that is bound to a defined area (site T₁₀₀) on the surface. The nucleic acid oligomer is added to the sample in an amount that is greater than the amount of nucleic acid oligomer necessary to completely associate the modified nucleic acid oligomers of site T₁₀₀. The last step of that method is to compare the values obtained by the detection of the fluorescence of the fluorophore with the value obtained for the area T₁₀₀. The value obtained for the area T₁₀₀ thus corresponds to the value at complete association (100%).

According to a particularly preferable embodiment a modified surface is used, which was modified by attaching at least three types of modified nucleic acid oligomers. The different types of modified nucleic acid oligomers are bound to the surface in spatial basically separated areas. Thereby at least one type of modified nucleic acid oligomer is attached to the surface in a defined area (site T₀), of which it is known that the sample does not contain a binding partner with a high association constant, thus the corresponding nucleic acid oligomer does not occur in the sample. Also in this particularly preferable method a nucleic acid oligomer is added to the sample before the exposure of the sample to the modified surface, with the nucleic acid oligomer being a binding partner with a high association constant for a certain type of modified nucleic acid oligomer, that is bound to a defined area (site T₁₀₀) on the surface. The nucleic acid oligomer is added to the sample in an amount that is greater than the amount of nucleic acid oligomer necessary to completely associate the modified nucleic acid oligomers of site T₁₀₀. The last step of that method is to compare the values obtained by the detection of the fluorescence of the fluorophore with the value obtained for the area T₁₀₀ and with the value obtained for the area T₀. The value obtained for the area T₀ thus corresponds to the value at lacking association (0%).

According to a most particularly preferred embodiment of the method described above before the exposure of the sample to the modified surface at least one further type of nucleic acid oligomer is added to the sample, of which it is known that this nucleic acid oligomer is not contained in the original sample. This further type of nucleic acid oligomer has an association constant >0 for a type of modified nucleic acid oligomer that is attached to the surface in a defined area (site Tₙ). The nucleic acid oligomer is added to the sample in such an amount that after exposing the sample to the modified surface n % of the modified nucleic acid oligomers of site Tₙ are in the associated state. The last step of this method is to compare the values obtained at the detection of the fluorescence of the fluorophore with the values obtained for the area T₁₀₀, with the value obtained for the area T₀ and with the values obtained for the areas Tₙ.

The value obtained for a certain test site Tₙ thus corresponds to the value at n % associates of modified nucleic acid oligomers and target nucleic acid oligomers relating to the total number of modified nucleic acid oligomers of the respective type.

The amount of nucleic acid oligomers, which has to be brought into contact with the modified surface to cause a n % association of site Tₙ can be determined by those skilled in the art with simple routine testing. For that purpose e.g. after the detection of the values for T₀ and T₁₀₀ a calibrated measurement is carried out with determining the signal intensity of (different) detection label the modified nucleic acid oligomers and the target nucleic acid oligomers are equipped with. The intensity ratio of the target nucleic acid oligomer label signal to the modified nucleic acid oligomer label signal corresponds n %.

If a sufficient number of sites Tₙ is applied to the modified surface, a scaling graph of high accuracy can be recorded. The scaling of the measurements of the actual test sites using this scaling graph significantly enhances the reproducibility of the analyses by means of DNA chip technology.

It should be pointed out that finding a nucleic acid oligomer that is not contained in the sample, does not cause any problems, since even the largest genomes still provides a sufficient variety of non existent sequences. In the case that the non-existent sequence differs from a present sequence only by a single base the hybridization step has to be carried out under stringent conditions. Sequences are used preferentially that differ clearly, thus by more bases, from sequences present in the sample. Particularly good results are achieved, if for the test sites and the scaling test sites oligonucleotides are used with the same or at least a similar number of bases.

The present invention is further directed to a kit comprising a modified surface, whereupon the modification consists of the attachment of at least one type of modified nucleic acid oligomer and the nucleic acid oligomers are modified by attaching at least one type of fluorophore.

According to the invention the detection of the fluorescence is carried out after adjusting a defined salt concentration higher than 0.5 mol/l in the solution surrounding the modified nucleic acid oligomers. Preferred salt concentrations are in the ranges between 0.5 and 10 mol/l, between 1 and 10 mol/l, between 0,5 and 3 mol/l and particularly between 2 and 3 mol/l, because it was found out that in these ranges an especially large difference in the fluorescence intensity exists between the hybridized and the non hybridized nucleic acid oligomer. Thus a particularly reliable detection of the hybridization events is made possible.

The Quenching Surface

The term “surface” refers to any support material that is appropriate directly or after an adequate chemical modification to bear fluorophore derivatized nucleic acid oligomers that are covalently attached to the surface and whose fluorescence is reduced significantly (>10% of the expected fluorescence intensity of the fluorophore in the absence of the surface under otherwise identical conditions) near the surface (in approx. 1 to 50 Å distance to the surface) by fluorescence quenching (radiationless energy transfer between the fluorophore as emitter and surface as absorber). Particularly suitable as quenching surface material are gold and silver. The term surface is independent of the spatial dimensions and includes also nanoparticles (particles or cluster of a few singular up to several thousands of surface atoms or molecules). Additionally the surface may be present bound to a solid support like e.g. glass, metal or plastic.

Binding a Nucleic Acid Oligomer to the Surface

Methods for the attachment of nucleic acid oligomers to the surface are known to those skilled in the art. The attachment can take place e.g. covalently via amino, hydroxy, epoxy or carboxy groups of the support material with thiol, hydroxy, amino or carboxy groups naturally present on the nucleic acid oligomer or affixed thereto by derivatization. The nucleic acid oligomer can be linked to the surface atoms or molecules of a surface either directly or via a linker/spacer. Furthermore the nucleic acid oligomer can be coupled by the methods common for immuno assays as e.g. using biotinylated nucleic acid oligomers to form a noncovalent immobilization on surfaces modified with avidin or streptavidin. The chemical modification of the probe nucleic acid oligomers with a surface anchor group can already be introduced during the course of the automated solid phase synthesis or with separate reactions sequences. Thereby also the nucleic acid oligomer is linked directly or via a linker/spacer with the surface atoms or molecules of a surface of the type described above. This binding may be carried out in different ways known from background art (cf. e.g. Hartwich, G. METHOD FOR ELECTROCHEMICALLY DETECTING SEQUENCE-SPECIFIC NUCLEIC ACID-OLIGOMER HYBRIDISATION EVENTS (1999), WO 00/42217).

When attaching the nucleic acid oligomers to the surfaces care must be taken of a particularly important point. Basically especially suitable conditions prevail for the detection of the difference of the fluorescence intensity between hybridized nucleic acid oligomers and single stranded nucleic acid oligomers if the fluorophore is only in one of the states “hybridized” or “non-hybridized” as near as possible to the modified surface. As is known the extent of the quenching act alters with a larger (third to sixth) power of the distance between the quenching surface and the fluorophore. Such particularly suitable conditions can only be achieved by special binding techniques. When attaching the nucleic acid oligomers care must be taken that these are attached to the surface either without any further coadsorbate or, if a coadsorbate appears to be necessary, that this forms a layer on the surface as thin as possible. Either a direct attachment of the nucleic acid oligomer at the surface has to be carried out or a covering together with preferentially short-chained coadsorbates like e.g. short-chained thiols. Preferred are coadsorbates with a chain length of 1 to 30, preferred 1 to 20, particularly preferred 1 to 10, and more particularly preferred 1 to 5.

Particularly unfavorable in this context is an attachment of the nucleic acid oligomers in the form of a linkage surface-biotine-avidin-biotine-oligomer. With such a connection the fluorophore is always shielded from the surface by a very thick layer of biotine-avidin-biotine, which involves corresponding disadvantages for the detection of the fluorescence.

Fluorophore

As fluorophores commercially available fluorescent dyes such as e.g. Texas Red®, Rhodamin dyes, cyanine dyes like e.g. Cy3™, Cy5™, fluoresceine etc. (cf. Fluka, Amersham and Molecular Probes catalogue) are used.

Fluorescence Quenching

The deactivation of an electronically excited species via a radiationless process is referred to as fluorescence quenching. The deactivation can occur by collisions or also by radiationless energy transfer to metals. The released energy is dissipated as thermal energy. Gold is an example for a metal that has the ability of quenching fluorescence. The quenching shows a strong dependency on the distance of the fluorophore to the surface, which acts as a fluorescence quencher (inversely proportional to a larger (third to sixth) power of the distance). Therefore the effect of fluorescence quenching is measurable only at distances lower than 100 to 200 Å. In the range above approx. 200 Å further distance alterations no longer lead to a measurable increase of the fluorescence intensity.

### BRIEF DESCRIPTION OF THE DRAWINGS

The invention will be explained in greater detail below by reference to exemplary embodiments in association with the drawings, wherein

FIG. 1 shows a schematic diagram of the detection of nucleic acid oligomer hybridization events by modulation of the fluorescence quenching at quenching surfaces

FIG. 2 A: Measurement of the change in fluorescence intensity of a 20mer and a 30mer nucleic acid probe (single stranded) in dependence of the ionic strength (here: salt concentration) of the solution above the surface;

B: Measurement of the change in fluorescence intensity before and after the sequence-specific hybridization of a 30mer nucleic acid probe with the complementary strand (target) in dependence of the ionic strength (here: salt concentration) of the solution above the surface.

### LIST OF REFERENCE SIGNS

102: Fluorophore
201: Single stranded oligomer probe
202: Probe hybridized with target
203: Surface (e.g. gold)
204: Distance of the fluorophore to the gold surface before the hybridization
205: Distance of the fluorophore to the gold surface after the hybridization

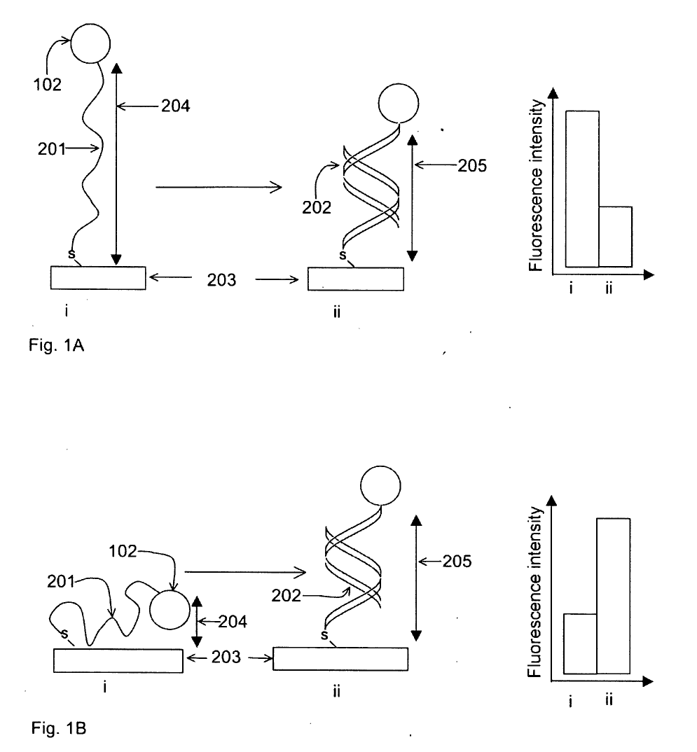

FIG. 1 shows a schematic diagram of the detection of nucleic acid oligomer hybridization events by modulation of the fluorescence quenching at quenching surfaces. In FIG. 1A it is illustrated that before hybridization the single stranded probe nucleic acid oligomer 201 is in a state, which is characterized by a large distance 204 between fluorophore 102, and quenching metal surface. By hybridization with the nucleic acid oligomer strand 202 (target) complementary thereto the distance 205 between the fluorescent dye molecule and the metal surface 203 acting as quencher decreases. This causes a decrease of the fluorescence intensity (bar graph of FIG. 1A). FIG. 1B illustrates the case that before hybridization the single stranded probe nucleic acid oligomer 201 is in a state, which is characterized by a low distance 204 between fluorophore 102, and quenching metal surface. By hybridization with the nucleic acid oligomer strand 202 (target) complementary thereto the distance 205 between the fluorescent dye molecule and the metal surface 203 acting as quencher increases. This causes an increase of the fluorescence intensity (bar graph of FIG. 1B).

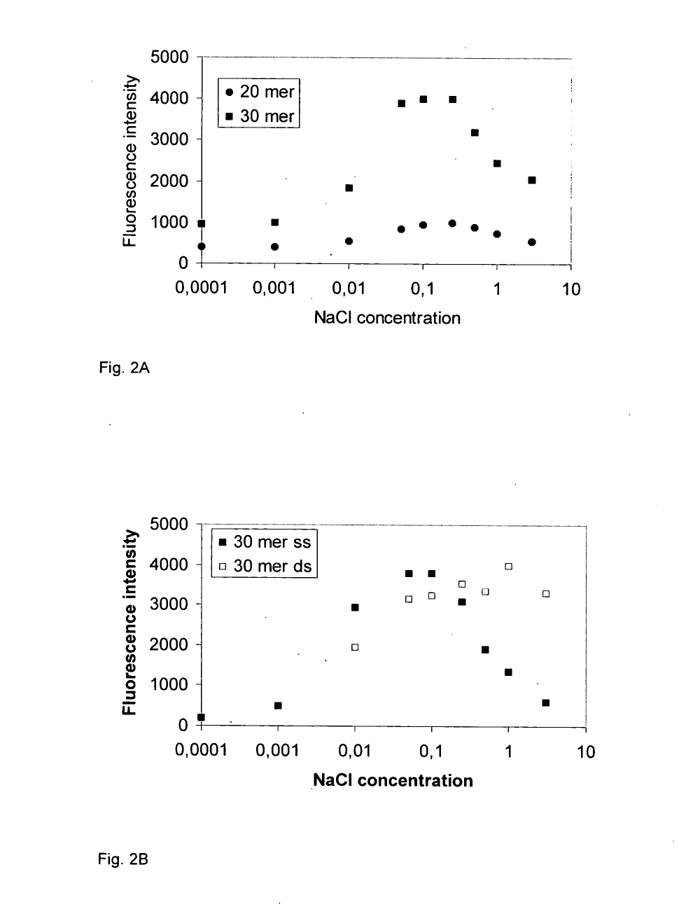

FIG. 2A shows a plot of the fluorescence intensity of a 20mer and a 30mer nucleic acid probe (single stranded) in dependence of the ionic strength (here: salt concentration) of the solution above the surface. The fluorescence intensity of 200 μm spots of single stranded probe oligonucleotides (20mer and 30mer) with Cy3™ as covalently attached fluorophore immobilized on a gold plate 1 cm² in size was measured. The plot according to FIG. 2B shows the fluorescence intensity before and after the sequence specific hybridization of a 30mer nucleic acid probe with the complementary strand (target) in dependency of the salt concentration above the solution under otherwise identical conditions as described in context of FIG. 2A. From FIG. 2B it is obvious that at salt concentrations above 0.5 mol/l the fluorescence intensity increases after hybridization by up to a factor of 5. This enables an unambiguous detection of the resulted hybridizations also for parallel methods.

### Manner of Executing the Invention

To use the advantages of the DNA chip technology for the detection of nucleic acid oligomer hybrids via modulation of fluorescence quenching different modified nucleic acid oligomer probes with different sequences are applied to a support. With the layout of the nucleic acid oligomer probes of known sequence on any position of the surface the hybridization event of any target nucleic acid oligomer or a (fragmented) target DNA should be detected in order to seek and sequence-specifically detect mutations in the target. For this purpose, the surface atoms or molecules of a defined are (a test site) on a surface are linked with DNA/RNA/PNA nucleic acid oligomers having a known but arbitrary sequence, as described above. In a most general embodiment the DNA chip may also be derivatized with a single probe nucleotide. Preferred probe nucleic acid oligomers are nucleic acid oligomers (e.g. DNA, RNA or PNA fragments) of base length 3 to 70, preferably of length 5 to 60, particularly preferably of length 10 to 50, more particularly preferably of length 12 to 40.

It should be mentioned here that the target oligonucleotides could also comprise a larger number of bases, thus be longer than the probe oligonucleotides. In this case the term “nucleic acid oligomer complementary to probe oligonucleotide” is understood to be a nucleic acid oligomer that has a base sequence, which is complementary to the probe oligonucleotide in a part. The remaining part/parts of the nucleic acid oligomer then protrude at the tail/tails of the probe oligonucleotide over its base chain.

On surfaces prepared in that way with immobilized and fluorophore marked probe oligonucleotides the fluorescence intensity of the fluorophore marked probe oligonucleotides in the single stranded state is determined e.g. with a fluorescence scanner by a reference measurement at a defined and preset salt concentration in the surrounding solution.

In the next step to the surface with immobilized probe oligonucleotides the assay solution of target oligonucleotides (as concentrated as possible) is added under for the hybridization stringent conditions. Thereby hybridization occurs only in the case that the solution contains target nucleic acid oligomer strands, which are complementary to the surface-bound probe nucleic acid oligomers, or complementary in at least wide areas.

After the hybridization between probe and target again a defined salt concentration is adjusted and then in a second fluorescence measurement the fluorescence intensity in the hybridized double stranded state is being determined.

For every test site the difference between reference and second measurement is proportional to the number of for the particular test site complementary (respectively complementary in wide areas) target oligonucleotides that had been originally present in the assay solution.

According to an alternative method the reference measurement can be omitted, if the value of the reference signal is sufficiently accurately known (e.g. from previous measurements etc.) beforehand.

As a result of the hybridization of the probe nucleic acid oligomer and the nucleic acid oligomer strand complementary thereto (target), the distance changes between the fluorescent dye molecule and the metal surface acting as quencher. Due to the altered distance also the degree of the quenching process and thus the intensity of the fluorescence undergoes a strong change. Thus a sequence specific hybridization event can be detected by fluorescence-based methods as e.g. fluorescence microscopy or measurements with fluorescence scanner.

Different topologies for the single stranded probe nucleic acid oligomer can be realized by a purposeful manipulation of the salt content of the solution:

a) Before the hybridization the single stranded probe nucleic acid oligomer 201 is in a state, which is characterized by a large distance 204 between fluorophore 102, and quenching metal surface (high fluorescence intensity). By hybridization with the nucleic acid oligomer strand 202 (target) complementary thereto the distance 205 between the fluorescent dye molecule and the metal surface 203 acting as quencher changes to that effect that by the decrease of the distance an augmentation of the quenching occurs and a lower fluorescence intensity can be observed after the hybridization (see FIG. 1A).

b) Before the hybridization the single stranded probe nucleic acid oligomer 201 is in a state, which is characterized by a low distance 204 between fluorophore 102 and the quenching metal surface (low fluorescence intensity). By hybridization with the nucleic acid oligomer strand 202 (target) complementary thereto the distance 205 between the fluorescent dye molecule and the metal surface 203 acting as quencher changes to that effect that by the increase of the distance a diminution of the quenching occurs and a higher fluorescence intensity can be observed after the hybridization (see FIG. 1B).

Both topologies shown above can be achieved by the variation of the ionic strength, particularly the salt concentration. Thereby any salts can be used with the exception of bivalent salts (e.g. Mg²⁺) or chaotropic salts. At low or medium salt content the surface-bound single stranded probe is in a more stretched conformation (see FIG. 1A). As a result of the hybridization a decrease of the fluorescence intensity is observed (see FIG. 1A, 2B). At high salt content the surface-bound single stranded probe is in a more compressed conformation (see FIG. 1B). As a result of the hybridization an increase of the fluorescence intensity is observed (see FIG. 1B, 2B).

### Embodiments

The nucleic acid probe n nucleotides long (DNA, RNA or PNA, e.g. an oligo 20 nucleotides long) is provided directly or via an (arbitrary) spacer with a reactive group near one of its ends (3′- or 5′-end) for the covalent linkage to the surface, e.g. as 3′-thiol modified probe oligonucleotide that uses the terminal thiol modification as reactive group for the attachment on gold. Further anchoring possibilities arise from e.g. an amino modified oligonucleotide that is used for a linkage to surface bound carboxylic acid functions (e.g. acid functionalized thiols as mercapto-propionic acid and an activation e.g. as activated ester. Near the other tail of the probe nucleotide a fluorophore is covalently attached (cf. Example 1). The thus modified nucleic acid probe is
(i) dissolved in buffer (e.g. 10-500 mM phosphate buffer, pH=7, 1 mM EDTA) brought into contact with the surface and there bound to the—where required adequately derivatized—surface via the reactive group of the probe nucleic acid oligomer
(ii) dissolved in buffer (e.g. 100 mM phosphate buffer, pH=7, 1 mM EDTA, 0,1-1 M NaCl) in the presence of a monofunctional linker brought into contact with the surface and there together with the monofunctional linker bound to the—where required adequately derivatized—surface via the reactive group of the probe nucleic acid oligomer with taking care that enough monofunctional linker of appropriate chain length is added (approximately 0.1 to 10 fold excess) to provide enough space between the individual probe oligonucleotides for the hybridization with the target oligonucleotides or
(iii) dissolved in buffer (e.g. 10-350 mM phosphate buffer, pH=7, 1 mM EDTA) brought into contact with the surface and there together with the monofunctional linker bound to the—where required adequately derivatized—surface via the reactive group of the probe nucleic acid oligomer. Subsequently the thus modified surface is brought into contact with a solution of the adequate monofunctional linker (e.g. alkane thiols or ω-hydroxy alkane thiols in phosphate buffer/EtOH mixtures with thiol modified probe oligonucleotides) with the monofunctional linker binding to the—where required adequately derivatized—surface via its reactive group (cf. paragraph “Binding a Nucleic Acid Oligomer to the Surface”).

The (residual) fluorescence of the fluorophore at the probe oligonucleotide is detected by a suitable method, e.g. by fluorescence measurement with a fluorescence scanner in presence of different salt concentrations (cf. Example 7). The fluorescence intensity of the single stranded probe oligonucleotide shows a maximum at a salt concentration between 0,05 and 0,25 mol/l (see FIG. 2A). Subsequently the dissolved target is added, potential hybridization events are enabled under appropriate conditions known by those skilled in the art (any arbitrary stringency conditions of the parameters potential/temperature (salt/chaotropic salts etc. for the hybridization) and the measurement for the detection of the fluorophore at a salt concentration accordant to the salt concentration of the first detection is repeated.

The difference in the measurement signal (decrease or increase respectively, depending on the salt concentration, cf. FIG. 1) is proportional to the number of hybridization events between a probe nucleic acid oligomer on the surface and the matching target nucleic acid oligomer in the assay solution (cf. Example 8)

The described method can be applied for one type of target (e.g. a certain type of target oligonucleotide with known sequence) on a surface or—using different type of probes for each test site respectively—for several types of target (several different types of target oligonucleotides).

### EXAMPLE 1

Producing the Amino-Modified Oligonucleotides for the Linkage as Probe Oligonucleotides on Modified Gold Surfaces

The synthesis of the oligonucleotides is carried out with an automated oligonucleotide synthesizer (Expedite 8909; ABI 384 DNA/RNA synthesizer according to the manufacturer's approved synthesis protocols for a 1.0 μmol synthesis. In the syntheses with the 1-O-Dimethoxytrityl-propyl-disulfid-CPG-carrier (Glen Research 20-2933) the oxidation steps are carried out using a 0.02 M iodine solution to prevent an oxidative cleavage of the disulfide bridge. Modifications at the 5′-position of the oligonucleotides are carried out in a to 5 min prolonged coupling step. The amino modifier C2 dT (Glen Research 10-1037) is incorporated in the sequences using the respective standard protocol. The coupling efficiencies are determined online during synthesis photometrically and conductometrically respectively via the DMT-cation concentration.

The oligonucleotides are deprotected with concentrated ammonia (30%) at 37° C. 16 h. The purification of the oligonucleotides is carried out using RP-HPL chromatography according to standard protocols (eluent: 0.1 M triethylammonium acetate buffer, acetonitril), characterized using MALDI-TOF MS. The amino-modified oligonucleotides are coupled to the appropriately activated fluorophores (e.g. fluorescein isothiocyanate) according to conditions known to those skilled in the art. The coupling can be carried out before as well as after the attaching the oligonucleotides to the surface.

### EXAMPLE 2

Producing the Fluorophore-Modified Oligonucleotides for the Linkage as Probe Oligonucleotides on Modified Gold Surfaces

The synthesis of the oligonucleotides is carried out with an automated oligonucleotide synthesizer (Expedite 8909; ABI 384 DNA/RNA synthesizer according to the manufacturer's approved synthesis protocols for a 1.0 μmol synthesis. In the syntheses with the 1-O-Dimethoxytrityl-propyl-disulfid-CPG-carrier (Glen Research 20-2933) the oxidation steps are carried out using a 0.02 M iodine solution to prevent an oxidative cleavage of the disulfide bridge. Modifications at the 5′-position of the oligonucleotides are carried out in a to 5 min prolonged coupling step. The fluorophores are incorporated as phosphoramidites (Glen Research 10-1037) during the last coupling step at the synthesizer. The coupling efficiencies are determined online during synthesis photometrically and conductometrically respectively via the DMT-cation concentration.

### EXAMPLE 3

Producing the Oligonucleotide Electrode Au—S(CH₂)₂-ss-oligo-FP

The quenching surface (here: gold plate) is incubated for 0.5-24 h with a doubly modified 20 bp single strand oligonucleotide having the sequence 5′-AGC GGA TAA CAC AGT CAC CT-3′ (modification 1: the phosphate group of the 3′ end is esterified with (HO—(CH₂)₂—S)₂ to form P—O—(CH₂)₂—S—S—(CH₂)₂—OH; modification 2: at the 5′ end the fluoresceine modifier fluoresceine-phosphoramidite (Proligo Biochemie GmbH) is incorporated according to the respective standard protocol) in a 5×10⁻⁵ molar buffer solution (phosphate buffer, 0.5 molar in water, pH 7) with addition of approx. 10⁻⁵ to 10⁻¹ molar propane thiol (or other thiols or disulfides of adequate chain length). During this reaction time the disulfide spacer P—O—(CH₂)₂—S—S—(CH₂)₂—OH of the oligonucleotide is homolytically cleaved. Thereby the spacer forms a covalent Au—S bond with Au atoms of the surface, thus causing a coadsorption of the ss-oligonucleotide and the cleaved 2-hydroxy mercaptoethanol. The free propane thiol that is also present in the incubation solution is likewise coadsorbed by forming an Au—S bond (incubation step). Instead of the single-strand oligonucleotide this single strand can also be hybridized with its complementary strand.

### EXAMPLE 4

Alternative Producing the Oligonucleotide Electrode Au—S(CH₂)₂-ss-oligo-FP

The alternative production of Au—S(CH₂)₂-ss-oligo-FP is composed in 2 sections, namely the derivatization of the gold surface with the probe oligonucleotide (incubation step) and the posttreatment of the thus modified electrode with an adequate bifunctional linker (posttreatment step).

The quenching surface (here: gold plate) is incubated for 0.5-24 h with a doubly modified 20 bp single strand oligonucleotide having the sequence 5′-AGC GGA TAA CAC AGT CAC CT-3′ (modification 1: the phosphate group of the 3′ end is esterified with (HO—(CH₂)₂—S)₂ to form P—O—(CH₂)₂—S—S—(CH₂)₂—OH; modification 2: at the 5′ end the fluoresceine modifier fluoresceine-phosphoramidite (Proligo Biochemie GmbH) is incorporated according to the respective standard protocol) in a 5×10⁻⁵ molar buffer solution (phosphate buffer, 0.5 molar in water, pH 7). During this reaction time the disulfide spacer P—O—(CH₂)₂—S—S—(CH₂)₂—OH of the oligonucleotide is homolytically cleaved. Thereby the spacer forms a covalent Au—S bond with Au atoms of the surface, thus causing a coadsorption of the ss-oligonucleotide and the cleaved 2-hydroxy mercaptoethanol. Instead of the single-strand oligonucleotide this single strand can also be hybridized with its complementary strand.

Subsequently the thus modified gold surface is completely wetted and incubated for 0.5-24 h with an approx. 10⁻⁵ to 10⁻¹ molar solution of short chained alkane thiols such as e.g. propane thiol in water or buffer, pH 7-7.5 or in ethanol). The free thiol covers the remaining free gold surface by forming an Au—S bond. Alternatively other functional thiols or disulfides of adequate chain length with the same or other functional groups can also be used.

### EXAMPLE 5

Measurement of the Fluorescence Intensity at the System Au-ss-oligo-fluoresceine and the System Au-ds-oligo-fluoresceine Respectively in Presence of Liquid Media

The probe surface is produced according to Example 4. For that purpose a modified oligonucleotide of the sequence 5′-fluoresceine-AGC GGA TAA CAC AGT CAC CT-3′ [C₃—S—S—C₃—OH] is immobilized on gold (50 μmol/l oligonucleotide in phosphate buffer (K₂HPO₄/KH₂PO₄ 500 mM, pH 7, posttreatment with propane thiol 1 mM in water) and the fluorescence intensity of the surface in the presence of different salt concentrations is determined in the form Au—S(CH₂)₂-ss-oligo-fluoresceine using a fluorescence scanner (Lavision Biotech). For the measurement of the fluorescence in the presence of liquid media 150 μl of the medium is added on top of the gold surface and subsequently covered with a cover slip. Alternatively hybriwells or imaging chambers can also be used.

### EXAMPLE 6

Modulation of the Distance by Means of the Ionic Strength/Salt Concentration

The probe is produced according to Example 4 and measured according to Example 5. By variation of the salt concentration (NaCl concentration) in a range between 1×10⁻⁴ und 3 mol/l the topology of the single stranded probe is modulated. The values obtained by measuring the fluorescence intensity in dependency of the salt concentration are illustrated in FIG. 2A. In the range of concentration between 0.01 and 0.5 mol/l, especially in the range between 0.05 and 0.25 mol/l the fluorescence intensity is maximum, i.e. the fluorophore has the largest distance to the gold surface.

### EXAMPLE 7

Fluorescence Measurement at the System Au-ss-oligo/dye-Modified Nucleic Acid Oligomers in the Absence and the Presence of Target Oligonucleotides (complementary to ss-oligo in Au-ss-oligo)

A probe electrode is produced according to Example 4. For that purpose a modified oligonucleotide of the sequence 5′-fluoresceine-AGC GGA TAA CAC AGT CAC CT-3′ [C₃—S—S—C₃—OH] is immobilized on gold (50 pmol/l oligonucleotide in phosphate buffer (K₂HPO₄/KH₂PO₄ 500 mM, pH 7). Subsequently according to Example 6 fluorescence measurements are carried out in the presence of NaCl solutions of different concentrations using a fluorescence scanner. After hybridization with complementary oligomers in phosphate buffer (500 mM, 1 mM EDTA, 1 M NaCl) according to Example 6 fluorescence measurements are carried out in the presence of NaCl solutions of different concentrations using a fluorescence scanner. The values in dependency of the salt concentration obtained at measuring the fluorescence intensity for the hybridized and the non-hybridized case are illustrated in FIG. 2B.

In the range above a salt concentration of 0.5 mol/l the fluorescence intensity is significantly higher after hybridization compared to before hybridization. This result is surprising, since the fluorescence of the single strand shows a maximum in the salt concentration range between 0.05 and 0.25 mol/l. The most significant difference in the fluorescence intensity before and after hybridization respectively appears however in a salt concentration range where the fluorescence of the single strand significantly decreases (FIG. 2B).

Die deutlichste Differenz der Fluoreszenzintensität vor bzw. nach Hybridisierung zeigt sich jedoch in einem Bereich der Salzkonzentration, in dem die Fluoreszenz des Einzelstrangs deutlich abnimmt (Figur 2B).

1. A method for detecting nucleic acid oligomer hybridization events by fluorescence quenching, comprising the steps
a) providing a modified surface, the modification comprising the attachment of at least one type of modified nucleic acid oligomers, wherein the nucleic acid oligomers (201) are modified by attaching of at least one type of fluorophore (102),
b) providing a sample having nucleic acid oligomers,
c) bringing the sample into contact with the modified surface,
d) adjusting a defined concentration of salt in the solution surrounding the modified nucleic acid oligomers, wherein a concentration of salt greater than 0.5 mol/l is set
e) detecting the fluorescence of the fluorophore (102),
f) comparing the fluorescence intensity detected in step e) with reference values

2. The method according to claim 1, wherein after step a) and before step c) the steps
b₃) adjusting a defined concentration of salt in the solution surrounding the modified nucleic acid oligomers, wherein the same concentration of salt being used as in step d) and
b₄) first detection of the fluorescence of the fluorophore (102)
are carried out and as step c) the step
c) adjusting stringency conditions for the hybridization and bringing the sample into contact with the modified surface
is carried out and in step f) the values obtained in step e) are compared with the reference values obtained in step b₄).

3. The method according to claims 1, wherein as step a) the step
a) providing a modified surface, the modification comprising the attachment of at least two types of modified nucleic acid oligomers (201), the different types of modified nucleic acid oligomers (201) being bound to the surface in spatially substantially separate regions, wherein the nucleic acid oligomers (201) are modified by attachment of at least one type of fluorophore,
is carried out and before step c) the steps
b₁) adding one type of nucleic acid oligomer to the sample, the type of nucleic acid oligomer being a binding partner having a high association constant of a type of modified nucleic acid oligomer bound to the surface in a specific region T₁₀₀, the nucleic acid oligomer being added in a quantity that is greater than the quantity of nucleic acid oligomers necessary to completely associate the modified nucleic acid oligomers of the T₁₀₀ site,
b₃) adjusting a defined concentration of salt in the solution surrounding the modified nucleic acid oligomers, the same concentration of salt being used as in step d) and
b₄) first detection of the fluorescence of the fluorophore (102)
are carried out and as step c) the step
c) adjusting stringency conditions for the hybridization and bringing the sample into contact with the modified surface
is carried out and in step f) the values obtained in step e) are compared with the value obtained in step b₄) for the T₁₀₀ region and with the reference values obtained in step b₄).

4. The method according to claim 3, wherein as step a) the step
a) providing a modified surface, the modification comprising the attachment of at least three types of modified nucleic acid oligomers (201), the differing types of modified nucleic acid oligomers are bound to the surface in spatially substantially separate regions, at least one type of modified nucleic acid oligomer (201) being attached to the surface in a specific region T₀, and no binding partner having a high association constant to said modified nucleic acid oligomer being contained in the sample, wherein the nucleic acid oligomers are modified by attachment of at least one type of fluorophore (102),
is carried out and in step f) the values obtained in step e) are compared with the value obtained in step b₄) for the T₁₀₀ region, with the value obtained in step b₄) for the T₀ region and with the reference values obtained in step b₄).

5. The method according to claim 3, wherein before step c) the step
b₂) adding of at least one additional type of nucleic acid oligomer to the sample, said type of nucleic acid oligomer not being contained in the sample provided in step b), and the nucleic acid oligomer exhibiting an association constant >0 to a type of modified nucleic acid oligomer that is bound to the surface in a specific region Tₙ, the nucleic acid oligomer being added in a quantity such that, after step c), n % of the modified nucleic acid oligomers in the Tₙ region are present in associated form
is carried out and in step f), the values obtained in step e) are compared with the value obtained in step b₄) for the T₁₀₀ region, with the value obtained in step b₄) for the T₀ region with the value obtained in step b₄) for the Tₙ region and with the reference values obtained in step b₄).

6. The method according to claim 1, wherein in step d) a concentration of salt between 0.5 and 10 mol/l, especially between 1 and 10 mol/l is set.

7. The method according to claim 6, wherein in step d) a concentration of salt between 0.5 and 3 mol/l is set.

8. The method according to claim 1, wherein the modified nucleic acid oligomers comprise 3 to 70 bases.

9. The method according to claim 1, the modification of the surface comprising the attachment exclusively of nucleic acid oligomers.

10. The method according to one of claim 1, wherein the surface is additionally modified by attachment of a short-chained coadsorbate.

11. A kit for carrying out a method according to claim 1, comprising a modified surface, the modification comprising the attachment of at least one type of modified nucleic acid oligomers, said nucleic acid oligomers being modified by the attachment of at least one type of fluorophore.
